(19) United States
(12) Patent Application Publication
(10) Pub. No.: US 2022/0305191 A1
(43) Pub. Date: Sep. 29, 2022

(54) FLUID COLLECTION SYSTEM INCLUDING A COMPRESSIBLE SUCTION DEVICE AND RELATED METHODS

(71) Applicant: PureWick Corporation, El Cajon, CA (US)

(72) Inventors: Dennis Joseph, Milton, GA (US); Hannah Rose Kriscovich, Marietta, GA (US); Jill W. Jones, Avondale Estates, GA (US); Juan Alejandro Saavedra Cisneros, Dacula, GA (US); David Xu, Cary, NC (US)

(21) Appl. No.: 17/655,464

(22) Filed: Mar. 18, 2022

Related U.S. Application Data
(60) Provisional application No. 63/165,384, filed on Mar. 24, 2021.

(51) Int. Cl. A61M 1/00

(52) U.S. Cl. CPC A61M 1/782; A61M 1/60; A61M 2039/242

(57) ABSTRACT

Examples relate to fluid collection systems and methods of use of the system. The fluid collection system may include a fluid collection device configured to receive fluid discharged from a user, a fluid collection container in fluid communication with the fluid collection device configured to collect the fluid and a suction device in fluid communication with the fluid collection device and the fluid collection container. The suction device is compressible and configured to draw fluid from the fluid collection device into the fluid collection container solely responsive to mechanical actuation of the suction device.

### CROSS-REFERENCE TO RELATED APPLICATIONS

This application claims priority to U.S. Provisional Patent Application No. 63/165,384 filed on Mar. 24, 2021, the disclosure of which is incorporated herein, in its entirety, by this reference.

### BACKGROUND

An individual may have limited or impaired mobility such that typical urination processes are challenging or impossible. For example, the individual may have surgery or a disability that impairs mobility. In another example, the individual may have restricted travel conditions such as those experience by pilots, drivers, and workers in hazardous areas. Additionally, fluid collection from the individual may be needed for monitoring purposes or clinical testing.

Bed pans and urinary catheters, such as a Foley catheter, may be used to address some of these circumstances. However, bed pans and urinary catheters have several problems associated therewith. For example, bed pans may be prone to discomfort, spills, and other hygiene issues. Urinary catheters be may be uncomfortable, painful, and may cause urinary tract infections. Conventional fluid collection devices also may be limited to use when a patient is confined to a chair or wheelchair.

Thus, users and manufacturers of fluid collection devices continue to seek new and improved devices, systems, and methods to collect bodily fluids such as urine.

### SUMMARY

Embodiments disclosed herein are related to fluid collection devices and methods of using fluid collection devices. In an embodiment, a fluid collection system is disclosed. The fluid collection system may include a fluid collection device configured to receive fluid discharged from a user and a fluid collection container in fluid communication with the fluid collection device configured to collect the fluid. The fluid collection system may further include a suction device in fluid communication with the fluid collection device and the fluid collection container. At least a portion of the suction device may be compressible and configured to draw fluid from the fluid collection device into the fluid collection container solely responsive to mechanical actuation of the suction device.

In an embodiment, a method of collecting urine discharged by a user is disclosed. The method may include receiving the urine discharged from the user in a fluid collection device. The method may also include drawing the urine from the fluid collection device into a suction device solely responsive to a mechanical actuation of the suction device. The suction device may include a first chamber, a second chamber, and a one-way valve disposed between the first chamber and the second chamber. The first chamber may receive the urine from the fluid collection device when the suction device is decompressed. The method may also include drawing the urine from the first chamber to the second chamber by compressing the suction device and discharging the urine from the second chamber into a fluid collection container.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is a schematic view of a fluid collection system, according to an embodiment.

FIG. 1B is an isometric view of a fluid collection system integrated with a wheelchair, according to an embodiment.

FIG. 2A is an isometric view of a fluid collection system integrated with a wheelchair, according to an embodiment.

FIG. 2B is a block diagram of an actuator and operator, according to an embodiment.

FIG. 2C is a block diagram of a controller in a fluid collection system, according to an embodiment.

FIG. 3A is a cross-sectional view of a suction device, according to an embodiment.

FIG. 3B is a cross-sectional view of a suction device, according to an embodiment.

FIG. 4 is a flow diagram of a method of collecting fluid discharged by a user, according to an embodiment.

### DETAILED DESCRIPTION

Embodiments disclosed herein are related to fluid collection devices and methods of using the same. The devices and systems disclosed herein are configured to collect fluids from an individual. The fluids collected by the fluid collection devices may include at least one of urine, vaginal discharge, penile discharge, reproductive fluids, blood, sweat, or other bodily fluids. Embodiments disclosed herein are related to chair or wheelchair mountable fluid collection systems and related methods. Many users of fluid collection devices are over 65 years old with limited mobility, often relying on wheelchairs as a primary mode of transportation. Many users also spend a significant amount of their day in a seated. Users and caregivers, then, are benefited from a fluid collection system that may be both discrete and mobile, allowing users to use the fluid collection system to collect fluid both at home and on the go.

In many embodiments described herein, a fluid collection system is compact and configured to be coupled to or mounted to a wheelchair. Embodiments of the fluid collection systems described herein may not rely on electricity or at least include portable electrical supply, allowing a user to be more mobile. In some embodiments, the fluid collection system also includes a controller having a programmable timer that may be configured to operate the system without direct user input.

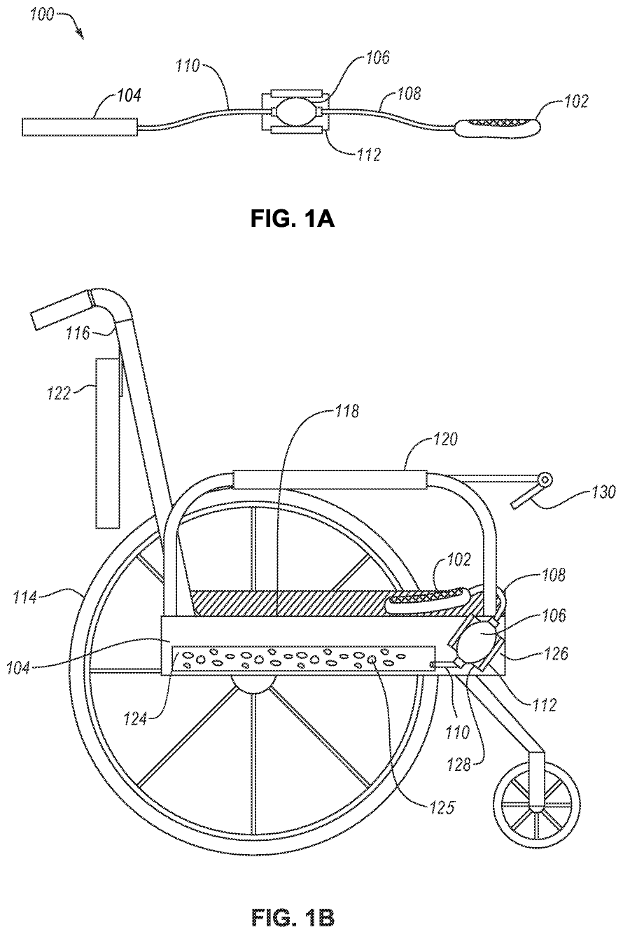

FIG. 1A is a schematic diagram of a fluid collection system 100, according to an embodiment. The fluid collection system 100 may be included in embodiments of fluid collection systems described herein. The system 100 includes a fluid (e.g., urine) collection device 102 (e.g., any of the fluid collection assemblies disclosed herein), a fluid collection container 104, and a suction device 106. The fluid collection device 102, the fluid collection container 104, and the suction device 106 may be fluidly coupled to each other via one or more tubes. For example, fluid collection device 102 may be in fluid communication to the suction device 106 via a first tube 108 and the suction device 106 may be in fluid communication with the fluid collection container 104 via a second tube 110. In some embodiments, the suction device 106 may be configured to receive fluid from the fluid collection device 102, the fluid may be drawn into and temporarily contained within the suction device 106, and then the suction device 106 may discharge the fluid to the fluid collection container 104. In some embodiments, fluid collection container 104 may be coupled to the suction device 106 via the second tube 110. In other embodiments, the suction device 106 may be disposed within the fluid collection container 104. Fluid (e.g., urine or other bodily fluids) collected in the fluid collection device 102 may be removed from the fluid collection device 102 via the first tube 108 coupled to the fluid collection device 102. Suction force may be introduced into the fluid collection device 102 via the first tube 108 responsive to suction (e.g., vacuum) force applied at the suction device 106.

The suction force may be applied to the suction device 106 by an actuator 112 operably coupled to the suction device 106. The suction device 106 may be configured to draw fluid from the fluid collection device 102 into the fluid collection container 104 solely responsive to mechanical actuation (e.g., compression) of the suction device 106. The suction force may be applied directly because at least a portion of the suction device 106 may be compressible. For example, the actuator 112 may apply a compression force on an exterior of the suction device 106 to maintain the suction device 106 in a compressed state. The actuator 112 may then release the compression force on the suction device 106 to decompress the suction device 106. The decompression of the suction device 106 may draw the fluid from the fluid collection device 102 and into an interior of the suction device 106 via the suction force created by the expansion of the interior of the suction device 106.

The fluid collection container 104 may be sized and shaped to retain a fluid therein. The fluid collection container 104 may include a bag (e.g., drainage bag), a bottle or cup (e.g., collection jar), or any other enclosed container for storing bodily fluid(s). The fluid collection container 104 may include an absorbent material. In some embodiments, the fluid collection container 104 may be decoupled from the fluid collection system 100 and be replaced. The fluid collection container 104 may include an odor neutralizer configured to at least partially neutralize the odor of the fluid. In some embodiments, the odor neutralizer may be included in the absorbent material.

The suction device 106 may include one or more of a manual vacuum pump, a bulb, a syringe, a tube, or any suitable compressible chamber configured to produce a vacuum. The suction device 106 may provide a vacuum or suction to remove fluid from the fluid collection device 102. The suction device 106 may include one or more suction devices 106. In some embodiments, the suction device may include one or more chambers. Some embodiments of the suction device 106 are described in more detail below.

In some examples, the actuator 112 may be a manual device. In some embodiments, at least a portion of the suction device 106 may be compressed directly by a user. In other embodiments, the actuator 112 may be powered by one or more of a power cord (e.g., connected to a power socket), one or more batteries, or an automatic controller or a remote operator. The remote operators disclosed herein may include one or more of a switch, a button, a lever, a remote, or any other device suitable to activate the actuator 112. In some examples, the actuator 112 may be sized and shaped to fit around or coupled to the suction device 106. In some embodiments, the actuator 112 may be configured to couple outside of, on, or within the fluid collection container 104. The actuator may include one or mechanical devices operating in combination. In some embodiments, the actuator 112 may include a pump.

Many embodiments of fluid collection systems described herein are securable or mountable to a chair or wheelchair. Turning to FIG. 1B, fluid collection system 100 is shown secured or mounted to a wheelchair 114. The wheelchair 114 may include any of a number of different conventional wheelchairs, and may include a back 116, a seat 118 and two arms 120. In some embodiments, the wheelchair 114 may include a pouch 122 on the back 116 of the wheelchair 114 that may support a storage system for the fluid collection system 100. For example, fluid collection containers 104 not currently coupled to the fluid collection system 100 may be stored within the pouch 122. The pouch 122 may be fixedly or removably secured to the back 116 of the wheelchair 114. For example, the pouch 122 may include a pack or bag having one or more straps configured to hang from or otherwise couple to the wheelchair 114.

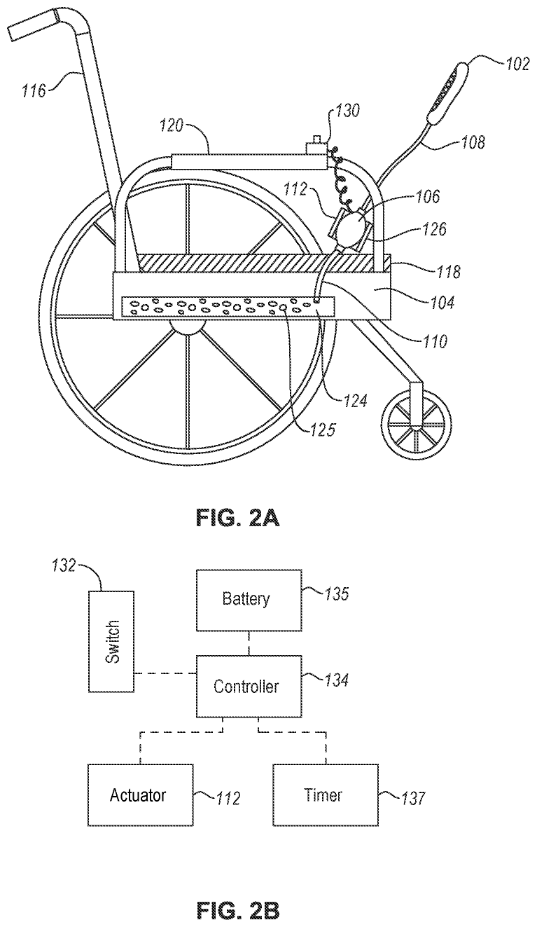

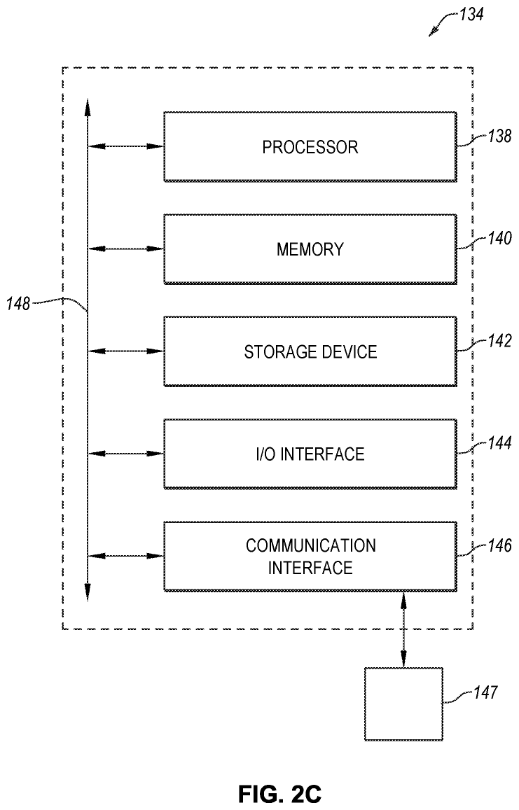

The fluid collection system 100 may include the fluid collection device 102. The fluid collection device 102 may include a male or female fluid collection device configured to be positioned at least proximate to a urethra of a user. PCT International Application No. PCT/US2019/029616 describes various embodiments of both male and female fluid collection devices, the disclosure of which is incorporated in its entirety by this reference. Moreover, the fluid collection device 102 may be interchangeable in the fluid collection system 100 between different types, varieties, and sizes of male or female fluid collection devices. The fluid collection assemblies shown in FIGS. 1A-2A are examples of female fluid collection assemblies that are configured to collect fluid(s) from females (e.g., collect urine from a female urethra). Further examples of female fluid collection assemblies are disclosed in U.S. Pat. No. 10,390,989 issued on Aug. 27, 2019, the disclosure of which is incorporated herein, in its entirety, by this reference. However, the fluid collection assemblies, systems, and methods disclosed herein may include male fluid collection assemblies and/or devices shaped, sized, and otherwise configured to collect fluid(s) from males (e.g., collect urine from a male urethra). Examples of male fluid collection assemblies are disclosed in U.S. Provisional Patent Applications No. 63/067,542 filed on Aug. 19, 2020 and U.S. patent application Ser. No. 16/433,773 filed on Jun. 6, 2019, the disclosure of which are incorporated herein, in its entirety, by this reference. Generally, the fluid collection device 102 may include a surface sized to be positioned proximate or adjacent to the urethra and configured to wick fluid or other fluids away from the user. Fluid or other fluids may be wicked from the surface to a reservoir in the fluid collection device 102.

The fluid collection system 100 also includes a first tube 108 in fluid communication with an interior region (e.g. reservoir) of the fluid collection device 102 and an interior region of the suction device 106. In some embodiments, as previously discussed, the fluid collection system 100 also may include a second tube 110 providing fluid communication between the suction device 106 and an interior region of the fluid collection container 104. In some embodiments, the suction device 106 may be disposed within the fluid collection container 104, and the second tube 110 may be absent from the fluid collection system 200 (as shown in FIG. 1B). The first tube 108 and/or the second tube 110 may include a flexible tube. In some embodiments, at least a portion of the first tube 108 and/or the second tube 110 may be substantially opaque, thereby inhibiting viewing of the fluid within the first tube 108 and the second tube 110.

In some embodiments, the fluid collection container 104 may be configured to be mounted underneath the seat 118 of the wheelchair 114. As shown in FIG. 1B, the fluid collection container 104 may include an interior region that stores fluid received from the fluid collection device 102 via the suction device 106. The fluid collection container 104 may be opaque or clear according to different embodiments. Fluid collected in the fluid collection container 104 may be emptied periodically by the user or a caregiver. The fluid collection container 104 may be reusable and dishwasher safe, and may include a generally rigid material such as polycarbonate or glass. In some embodiments, the fluid collection container 104 may be disposable and/or replaceable.

In some embodiments, the fluid collection container 104 includes an interior region sized and dimensioned to contain the fluid drawn from the fluid collection device 102. In some embodiments, the fluid collection container 104 may hold about 1 liter to about 3 liters, about 1 liter, about 2 liters, or about 3 liters of the fluid therein. The fluid collection container 104 may be generally enclosed and considered leak-proof. For example, the fluid collection container 104 may include a sealed plastic bag or a rigid plastic container. The fluid collection container 104 includes an absorbent material 124 disposed therein. In some embodiments, the absorbent material 124 may receive and hold therein the bodily fluid drawn from the fluid collection device 102. The absorbent material 124 may be integrated into the fluid collection container 104 and, thus, a user or caregiver may insert and/or remove the fluid collection container 104 that includes the absorbent material 124. As such, a soiled absorbent material 124 may indicate when the fluid collection container 104 should be removed from the fluid collection system 100 and replaced with a fresh fluid collection container 104. In some embodiments, the absorbent material 124 may fill at least a portion of the interior of the fluid collection container 104.

In some embodiments, the absorbent material 124 may include a synthetic absorbent, such as polyester or any other suitable hydrophilic absorbent material. In some embodiments, the absorbent material may include woven or non-woven sheet material, cellulosic pulp, synthetic pulp, pulp fibers, sponge material, or other suitable synthetic or natural materials. In other embodiments, the absorbent material 124 may include beads configured to expand and/or form a hydrogel. In some embodiments, the absorbent material 124 may be woven into an absorbent fabric. In some embodiments, the absorbent material 124 may include a biodegradable, a non-biodegradable material, or a combination of a biodegradable and a non-biodegradable material. In some embodiments, the bead structures of absorbent material 124 may be converted into a gel-like state as it absorbs fluid. In other embodiments, the absorbent material 124 may include a diaper-like absorbent material that converts the material and fluid into a gel-like state as it is collected and absorbed. The fluid collection container 104 may further include an odor neutralizer 125 configured to at least partially neutralize the odor of the fluid. In some embodiments, the odor neutralizer 125 may be integrated into the absorbent material 124. The fluid collection container 104 may include any suitable odor neutralizing agent such as synthetic or natural odor neutralizers. For example, the odor neutralizer may include vitamin C, baking soda, vinegar, and/or essential oils.

In some embodiments, the fluid collection container may be configured to be mounted underneath the seat 118 of the wheelchair 114. The fluid collection container 104 may be sized and dimensioned to fit underneath the seat 118 of the wheelchair 114. In some embodiments, the fluid collection container 104 may be generally rectangular (e.g., rectangular cuboid or right rectangular prism). The suction device 106 and/or the actuator 112 may be attached to and/or positioned proximate to or disposed within the fluid collection container 104. When the suction device 106 and the actuator 112 are coupled to the fluid collection container 104, the fluid collection container 104, the suction device 106, and the actuator 112 may together form a generally rectangular profile and be configured to fit under the seat of the wheelchair 114. In some embodiments, the fluid collection container 104 may be configured or positioned below the suction device 106. Thus, when the fluid is discharged from the suction device 106, the fluid may drain from the suction device 106 into the fluid collection container 104 via gravity.

The actuator 112 may be operably coupled to the suction device 106. In some embodiments, the actuator 112 may be configured to compress and decompress the suction device 106. The actuator 112 may include a compression system similar to a caliper brake system. The actuator 112 may include at least one moveable arm 126 coupled to a frame 128 proximate to the suction device 106, such that the suction device 106 may be disposed within the actuator 112. In an embodiment, the arm 126 may proximate and/or surrounding the suction device 106. In some embodiments, two arms 126 may be disposed on either side of the suction device 106. The arm and/or arms 126 may pivot when the actuator 112 is activated to either compress or decompress the suction device 106. However, any suitable mechanism configured to compress and decompress the suction device 106 may be utilized. In other embodiments, a mechanical press, a hydraulic system, a peristaltic pump, or a simple lever system or clamp may be included.

In some embodiments, the actuator 112 may be operably coupled to an operator 130. The operator 130 may be configured such that a user or caretaker may control the suction device 106 while either on the wheelchair or assisting the user. In some embodiments, the operator 130 may be located on an arm 120 of the wheelchair 114. In some embodiments, the operator may be a mechanical device such as a lever. For example, a user may pull the lever and the actuator 112 releases the suction device 106 and causes the suction device to decompress. The decompression of the suction device 106 may cause a vacuum to draw the fluid from the fluid collection device 102. The vacuum may cause the fluid to flow into the suction device 106. The fluid may then be temporarily contained within the suction device 106. The user may then push the lever causing the actuator 112 to compress the suction device 106 causing the fluid to discharge to the fluid collection container 104.

Referring now to FIG. 2A-2B, in an embodiment, the operator may include a switch 132 operatively coupled to the actuator 112. In other embodiments, the operator 130 may include a button, a remote, or any other suitable device configured to operate the actuator 112. In some embodiments, the actuator also includes a controller 134. The controller 134 may be included with the actuator 112 or the operator 130 or may be a separate component configured to communicate with the actuator 112. The controller 134 may include a communication interface configured to send notifications or alerts to other electronic devices. For example, the communication interface may be configured to send notifications or alerts at a selected radio frequency, via BLUETOOH, or via WI-FI to another electronic device, such as a smartphone of the user or caregiver. In some embodiments, the controller may be configured to send a text message or other alert to the smartphone or other electronic device of the user or caregiver when fluid in the fluid collection container 104 is at the predetermined threshold or a there may be a malfunction in the fluid collection system. The controller 134 may be powered by an external or internal battery 135, such as a rechargeable battery. The controller may wirelessly transmit an alert to the electronic device of the user or the caregiver when the battery 135 powering at least one of the controller, a pump, or other component is low. In some embodiments, the controller 134 may include a programmable timer 137. In some embodiments, the programmable timer 137 may be configured to operate the actuator 112 according to a preprogrammed schedule. Thus, the fluid collection system 100 may continue to operate without user involvement (e.g. when the user is sleeping or incapacitated).

FIG. 2C provides additional details of controller 134. FIG. 2C is a schematic of controller 134. The controller 134 may be configured to implement any of the example acts or steps disclosed herein, such as wired or wirelessly communicating with the actuator 112, the operator 130, or other electronic devices.

The controller 134 includes at least one computing device 136, according to an embodiment. The at least one computing device 136 is an exemplary computing device that may be configured to perform one or more of the acts described above. The computing device 136 may comprise at least one processor 138, memory 140, a storage device 142, an input/output (“I/O”) device/interface 144, and a communication interface 146. While an example computing device 136 is shown in FIG. 2C, the components illustrated in FIG. 2C are not intended to be limiting of the controller 134 and/or computing device 136. Additional or alternative components may be used in some examples. Further, in some examples, the controller 134 or the computing device 136 may include fewer components than those shown in FIG. 2C. For example, the controller 134 may not include the one or more additional computing devices 136. In some examples, the at least one computing device 136 may include a plurality of computing devices. Components of computing device 136 shown in FIG. 2C are described in additional detail below.

In some examples, the processor(s) 138 may include hardware for executing instructions (e.g., instructions for carrying out one or more portions of any of the methods disclosed herein), such as those making up a computer program. For example, to execute instructions, the processor(s) 138 may retrieve (or fetch) the instructions from an internal register, an internal cache, the memory 140, or storage device 142 and decode and execute them. In particular examples, processor(s) 138 may include one or more internal caches for data such as tables pertaining to volumetric tables. As an example, the processor(s) 138 may include one or more instruction caches, one or more data caches, and one or more translation lookaside buffers (TLBs). Instructions in the instruction caches may be copies of instructions in memory 140 or storage device 142. In some examples, the processor 138 may be configured (e.g., include programming stored thereon or executed thereby) to carry out one or more portions of any of the example methods disclosed herein.

In some examples, the processor 138 may be configured to perform any of the acts disclosed herein or cause one or more portions of the computing device 136 or controller 134 to perform at least one of the acts disclosed herein. Such configuration may include one or more operational programs (e.g., computer program products) that are executable by the at least one processor 138. For example, the processor 138 may be configured to automatically determine the proper programming schedule to operate the actuator 112 and/or automatically transmit an alert when a change or recharge of battery is suggested.

The at least one computing device 136 (e.g., a server) may include at least one memory storage medium (e.g., memory 140 and/or storage device 142). The computing device 136 may include memory 140, which is operably coupled to the processor(s) 138. The memory 140 may be used for storing data, metadata, and programs for execution by the processor(s) 138. The memory 140 may include one or more of volatile and non-volatile memories, such as Random Access Memory (RAM), Read Only Memory (ROM), a solid state disk (SSD), Flash, Phase Change Memory (PCM), or other types of data storage. The memory 140 may be internal or distributed memory.

The computing device 136 may include the storage device 142 having storage for storing data or instructions. The storage device 142 may be operably coupled to the at least one processor 138. In some examples, the storage device 142 may include a non-transitory memory storage medium, such as any of those described above. The storage device 142 (e.g., non-transitory storage medium) may include a hard disk drive (HDD), a floppy disk drive, flash memory, an optical disc, a magneto-optical disc, magnetic tape, or a Universal Serial Bus (USB) drive or a combination of two or more of these. Storage device 142 may include removable or non-removable (or fixed) media. Storage device 142 may be internal or external to the computing device 136. In some examples, storage device 142 may include non-volatile, solid-state memory. In some examples, storage device 142 may include read-only memory (ROM). Where appropriate, this ROM may be mask programmed ROM, programmable ROM (PROM), erasable PROM (EPROM), electrically erasable PROM (EEPROM), electrically alterable ROM (EAROM), or flash memory or a combination of two or more of these. In some examples, one or more portions of the memory 140 and/or storage device 142 (e.g., memory storage medium(s)) may store one or more databases thereon.

The computing device 136 also includes one or more I/0 devices/interfaces 144, which are provided to allow a user to provide input to, receive output from, and otherwise transfer data to and from the computing device 136. These I/O devices/interfaces 144 may include a mouse, keypad or a keyboard, a touch screen, camera, optical scanner, network interface, web-based access, modem, a port, other known I/O devices or a combination of such I/O devices/interfaces 144. The touch screen may be activated with a stylus or a finger.

The I/O devices/interfaces 144 may include one or more devices for presenting output to a user, including, but not limited to, a graphics engine, a display (e.g., a display screen or monitor), one or more output drivers (e.g., display drivers), one or more audio speakers, and one or more audio drivers. In certain examples, I/O devices/interfaces 144 may be configured to provide graphical data to a display for presentation to a user. The graphical data may be representative of one or more graphical user interfaces and/or any other graphical content as may serve a particular implementation.

The computing device 136 may further include a communication interface 146. The communication interface 146 may include hardware, software, or both. The communication interface 146 may provide one or more interfaces for communication (such as, for example, packet-based communication) between the computing device 510 and one or more additional computing devices 512 or one or more networks. For example, communication interface 146 may include a network interface controller (NIC) or network adapter for communicating with an Ethernet or other wire-based network or a wireless NIC (WNIC) or wireless adapter for communicating with a wireless network, such as a WI-FI. The one or more additional computer device 147 may include a smart phone of the user, a smart phone of the caregiver, an electronic device on the wheelchair, and/or a computer device of a healthcare system.

Any suitable network and any suitable communication interface 136 may be used. For example, computing device 136 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, one or more portions of controller 134 or computing device 136 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination thereof. The computing device 136 may include any suitable communication interface 146 for any of these networks, where appropriate.

The computing device 136 may include a bus 148. The bus 148 may include hardware, software, or both that couples components of computing device 136 to each other. For example, bus 148 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination thereof.

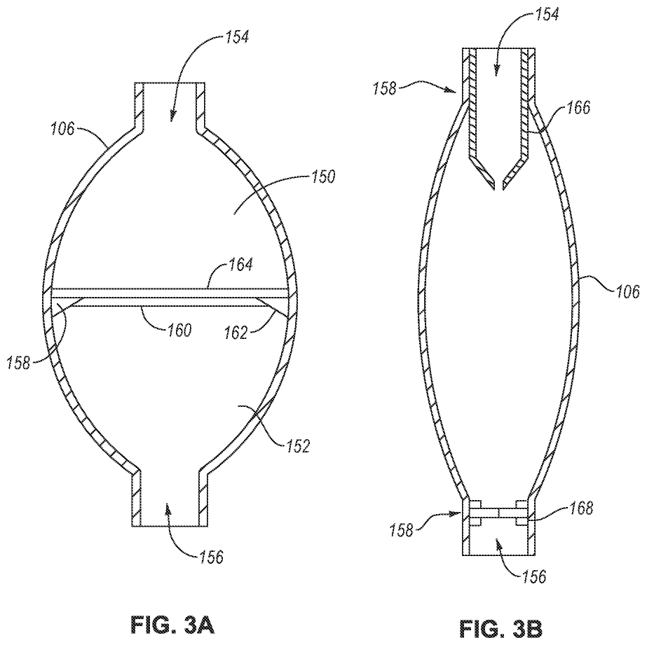

FIG. 3A is a cross-sectional view of suction device 106 according to at least one embodiment. The fluid may be drawn into and temporarily contained within the suction device 106. In some embodiments, the suction device 106 may include a first chamber 150 and a second chamber 152. The first chamber 150 may include an inlet 154 to receive the fluid from the fluid collection device 102 and the second chamber 152 may include an outlet 156 to expel the fluid to the fluid collection container 104. The suction device 106 may include a one-way valve 158 disposed between the first chamber 150 and the second chamber 152.

In some embodiments, the one-way valve 158 may include at least one of a ball check valve, a cross-slit valve, a duckbill valve, or a diaphragm valve (as shown in FIG. 3A). The flow of fluid through the suction device 106 is configured to be from the inlet 154 to the outlet 156. The one-way valve may be configured to allow fluid to flow from the inlet 154 to the outlet 156 and prevent flow from the outlet 156 to the inlet 154. The one-way valve 158 may limit spills or leaks and help control odors from the fluid collection system 100. At least a portion of the one-way valve 158 may be configured to be compressible and resilient. In some embodiments, the compression of at least a portion of the suction device 106 may be configured to open the one-way valve 158. The one-way valve 158 may be opened and/or shut by the pressure difference between the first chamber 150 and the second chamber 152. The one-way valve 158 may be configured to be normally opened or normally closed. In some embodiments, a self-centering flexible elastomer disc 160 may be included in the one-way valve 158. The valve body 162 may be configured to maintain the disc 160 centered over the seating area 164 of the one-way valve 158. In some embodiments, the seating area 164 may be on the side of the one-way valve 158 facing the inlet 154. In other words, the seating area 164 may be proximate the first chamber 150. The closed disc 160 prevents a backflow from the second chamber 152 to the first chamber 150. In some embodiments, suction pressure between the second chamber 152 and the first chamber may cause the disc 160 to unseat, and the fluid may flow around the disc 160 and into the second chamber 152.

Referring now to FIG. 3B, in some embodiments, the suction device 106 may include the inlet 154 in fluid communication with the fluid collection device 102 and the outlet 156 in fluid communication with the fluid collection container 104. The one-way valve 158 may be disposed within the inlet 154 or the outlet 156. In some embodiments, when the one-way valve 158 is disposed within the inlet 154 or the outlet 156, the first chamber 150 or the second chamber 152 may include the first tube 108 or the second tube 110. In some embodiments, the one-way valve 158 may be a first valve 166. The first valve 166 may be disposed within the inlet 154. The first valve 166 may be a ball check valve, a cross-slit valve, a duckbill valve, a diaphragm valve, or any other suitable one-way valve. In some embodiments, the suction device 106 may include a second valve 168. The second valve 168 may be disposed within the outlet 156. In some embodiments, the second valve 168 may also be a ball check valve, a cross-slit valve, a duckbill valve, a diaphragm valve, or any other suitable one-way valve as described above. In some embodiments, the first valve 166 and/or the second valve 168 may be configured to allow air to pass through from the second chamber 152 into the first chamber 150 but not allow fluids. In some embodiments, the first valve 166 may include a different type of one-way valve 158 than the second valve 168. In FIG. 3B, for example, the first valve 166 is shown as a duckbill valve and the second valve 168 is shown as a cross-slit valve. In some embodiments, at least a portion of the first valve 166 and or/the second valve 168 may be configured to be compressible and resilient.

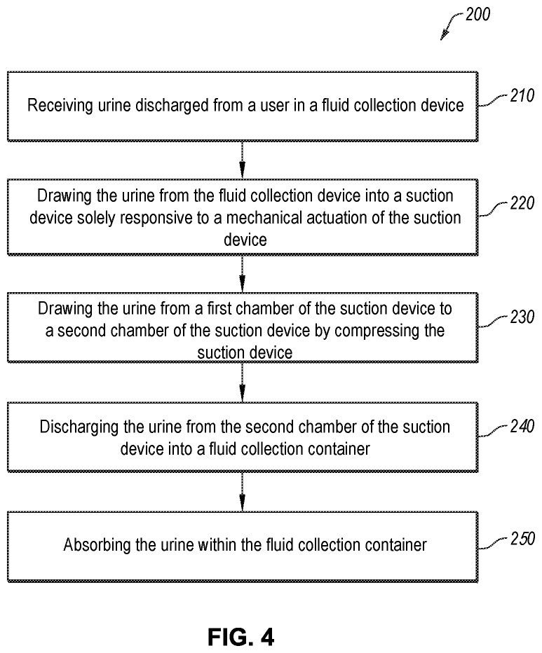

FIG. 4 is a flow diagram of a method 200 for collecting urine discharged by a user, according to an embodiment. The method 200 may include an act 210 of receiving the urine discharged from the user in a fluid collection device positioned at least proximate to a urethra of the user. The method 200 may also include an act 220 of drawing the fluid from the fluid collection device into a suction device solely responsive to a mechanical actuation of the suction device. The suction device may include a first chamber, a second chamber, and a one-way valve disposed between the first chamber and the second chamber. The first chamber may receive the urine from the fluid collection device when the suction device is decompressed. In some embodiments, the method 200 may include an act 230 of drawing the urine from the first chamber to the second chamber by compressing the suction device. The method may also include an act 240 of discharging the urine from the second chamber of the suction device into a fluid collection container. In some embodiments, the fluid collection container may include an absorbent material. The method 200 may also include an act 250 of absorbing the urine within the fluid collection container.

The mechanical actuator of the suction device may include an act of operating an actuator to decompress the suction device causing a vacuum effective to draw the urine from the fluid collection device into the suction device and operating the actuator again to compress the suction device causing the urine within the suction device to discharge into the fluid collection container. In some embodiments, the actuator may include a controller having a programmable timer that is configured to control the actuator. The method may also include programming the timer such that the fluid may be discharged from the user in a fluid collection device without requiring a direct action from the user.

The acts of the method 200 described above are for illustrative purposes. For example, the acts of the method 200 can be performed in different orders, split into multiple acts, modified, supplemented, or combined. In an embodiment, one or more of the act of the method 200 can be omitted from the method 200. Any of the acts of the method 200 can include using any of the fluid collection systems disclosed herein.

As used herein, the term “about” or “substantially” refers to an allowable variance of the term modified by “about” or “substantially” by ±10% or ±5%. Further, the terms “less than,” “or less,” “greater than,” “more than,” or “or more” include, as an endpoint, the value that is modified by the terms “less than,” “or less,” “greater than,” “more than,” or “or more.”

While various aspects and embodiments have been disclosed herein, other aspects and embodiments are contemplated. The various aspects and embodiments disclosed herein are for purposes of illustration and are not intended to be limiting.

What is claimed is:

1. A fluid collection system, comprising:
a fluid collection device configured to receive fluid discharged from a user;
a fluid collection container in fluid communication with the fluid collection device configured to collect the fluid; and
a suction device in fluid communication with the fluid collection device and the fluid collection container, wherein at least a portion of the suction device is compressible and configured to draw fluid from the fluid collection device into the fluid collection container solely responsive to mechanical actuation of the suction device.

2. The fluid collection system of claim 1, further comprising:
a first tube in fluid communication with the fluid collection device, wherein the suction device is coupled to the first tube and the tube is configured to receive fluid from the fluid collection device; and
a second tube in fluid communication with the fluid collection container, wherein the suction device is coupled to the second tube and is configured to discharge fluid to the fluid collection container.

3. The fluid collection system of claim 1, wherein the fluid is drawn into and temporarily contained within the suction device.

4. The fluid collection system of claim 1, further comprising an actuator operably coupled to the suction device, the actuator configured to compress the suction device and enable decompression of the suction device.

5. The fluid collection system of claim 4, wherein the actuator is operably coupled to an operator located on an arm of a wheelchair.

6. The fluid collection system of claim 4, further comprising a controller having a programmable timer that is configured to control the actuator.

7. The fluid collection system of claim 1, wherein the suction device includes a first chamber and a second chamber, wherein the first chamber receives the fluid from the fluid collection device and the second chamber includes an opening to expel the fluid to the fluid collection container.

8. The fluid collection system of claim 7, wherein the suction device includes a one-way valve disposed between the first chamber and the second chamber.

9. The fluid collection system of claim 8, wherein the one-way valve includes at least one of a ball check valve, a cross-slit valve, a duckbill valve, or a diaphragm valve.

10. The fluid collection system of claim 1, wherein the suction device includes an inlet in fluid communication with the fluid collection device and an outlet in fluid communication with the fluid collection container, wherein a one-way valve is disposed within the inlet or the outlet.

11. The fluid collection system of claim 10, wherein the one-way valve is a first valve disposed within the inlet, and the suction device includes a second valve disposed within the outlet.

12. The fluid collection system of claim 1, wherein the fluid collection container is configured to be mounted underneath a seat of a wheelchair.

13. The fluid collection system of claim 1, wherein the suction device is disposed within the fluid collection container.

14. The fluid collection system of claim 1, wherein the fluid collection container is positioned below the suction device, wherein the fluid drains from the suction device into the fluid collection container via gravity.

15. The fluid collection system of claim 1, wherein the fluid collection container includes an absorbent material.

16. The fluid collection system of claim 15, wherein the absorbent material includes gel beads.

17. The fluid collection system of claim 1, wherein the fluid collection container includes an odor neutralizer configured to at least partially neutralize an odor of the fluid.

18. A method of collecting urine discharged by a user, the method including:
receiving the urine discharged from the user in a fluid collection device positioned at least proximate to a urethra of the user; and
drawing the urine from the fluid collection device into a suction device solely responsive to a mechanical actuation of the suction device, wherein the suction device includes a first chamber, a second chamber, and a one-way valve disposed between the first chamber and the second chamber, wherein the first chamber receives the urine from the fluid collection device when the suction device is decompressed;
drawing the urine from the first chamber to the second chamber by compressing the suction device; and
discharging the urine from the second chamber into a fluid collection container.

19. The method of claim 18, wherein the fluid collection container includes an absorbent material, the method further comprising absorbing the urine within the fluid collection container.

20. The method of claim 18, wherein the mechanical actuation of the suction device includes operating an actuator to decompress the suction device causing a vacuum effective to draw the fluid from the fluid collection device into the suction device and operating the actuator again to compress the suction device causing the fluid within the suction device to discharge into the fluid collection container.

21. The method of claim 20, wherein the actuator includes a controller having a programmable timer that is configured to control the actuator.
